(19) United States
(12) Patent Application Publication
(10) Pub. No.: US 2025/0134792 A1
(43) Pub. Date: May 1, 2025

(54) COMPOSITIONS AND METHODS FOR TREATING SKIN

(71) Applicant: L'Oreal, Paris (FR)

(72) Inventors: Rebecca BARRESI, Robbinsville, NJ (US); Charbel Bouez, Hoboken, NJ (US); Carine Ballihaut, Aulnay-sous-Bois (FR); Elena Catalan Martin, Paris (FR); Franck Juchaux, Chevilly-Larue (FR); Julien Laboureau, Chevilly-Larue (FR); Jinyuan Yan, Clark, NJ (US)

(21) Appl. No.: 18/498,706

(22) Filed: Oct. 31, 2023

(51) Int. Cl. A61K 8/60; A61K 8/67; A61Q 19/00

(52) U.S. Cl. CPC A61K 8/602; A61K 8/675; A61Q 19/00; A61K 2800/81

(57) ABSTRACT

The disclosure relates to compositions and methods for improving the appearance of skin. The methods comprise treating skin tissue with at least one C-glycoside or derivative thereof, at least one vitamin B3 or derivative thereof and at least one skin modification stimulus, and the compositions comprise at least one C-glycoside or derivative thereof and/or at least one vitamin B3 or derivative thereof.

### TECHNICAL FIELD

The disclosure relates to compositions and methods for treating skin, for example for improving post-aesthetic skin procedure outcomes. The compositions comprise at least one C-glycoside or derivative thereof and/or at least one vitamin B3 or derivative thereof, and the methods comprise treating skin with at least one C-glycoside or derivative thereof, at least one vitamin B3 or derivative thereof, and at least one skin modification stimulus.

### BACKGROUND

Non-surgical aesthetic procedures that remove parts of the epidermis can accelerate the renewal of the skin's surface. For example, clinicians may use skin modification techniques such as laser treatments and chemical peels to improve the skin's appearance. Generally, skin treated with such lasers and peels go through stages of inflammation, granulation, and matrix remodeling during the post-procedure healing process. The inflammation stage begins shortly after the injury and lasts for several days, and the granulation stage begins about 24 hours after the injury and lasts for one to two weeks. The remodeling stage lasts up to about a year and involves collagen cross-linking and replacement, wherein a new collagen matrix forms.

The signaling pathway of prostaglandin E₂ (PGE₂) is of particular importance to skin inflammation. PGE₂ has been shown to be present at high levels in interleukin-1α (IL-1α) stimulated fibroblasts. Erythema and inflammation-associated hyperpigmentation caused by lasers and other resurfacing treatments is believed to be caused, in part, by local increases in reactive oxygen species (ROS) concentration, which stimulates mitogen-activated protein (MAP) kinases and the pro-inflammatory cyclooxygenase-2 (COX2), which in turn increases PGE₂ secretion. PGE₂ induces acute inflammation through mast cell activation via the EP3 receptor. PGE₂ also induces chronic inflammation.

Sulfated glycosaminoglycan (sGAG) release further mediates dermal regeneration, including wound contraction, after such skin procedures. SGAGs stimulate myofibroblasts in granulated tissue to close wounds by strong contractile forces caused in part by α-smooth muscle actin (α-SMA) contraction. Increased secretion of sGAGs in turn inhibits neutrophil elastase activity, signifying protection of the extracellular matrix (ECM). Ki-67 protein (also known as MKI67, has similarly been shown to be present during cell proliferation. Modifying the expression and secretion of these markers has the potential to improve skin healing.

Many individuals wish to enhance the outcome of their aesthetic procedures. Thus, there is an ongoing need for improved skin treatments that mediate these signaling pathways thereby reducing inflammation and skin discoloration and improving skin healing. The development of improved outcomes for aesthetic procedures is important to improve the quality of the results of such treatments and to avoid potential undesirable side effects such as skin discoloration.

It has now been surprisingly discovered that C-glycosides and derivatives thereof such as proxylane (also known as Pro-xylane™ and hydroxypropyl tetrahydropyrantriol), when combined with at least one vitamin B3 or a derivative thereof such as niacinamide, have an unexpected advantage in enhancing epidermal and dermal renewal, and are particularly effective in providing inflammation management and/or pigmentation benefits to the skin, for example following various skin procedures.

### SUMMARY

The present disclosure relates to methods for improving skin turnover and recovery post aesthetic procedure and compositions used therein. The combination of C-glycosides and derivatives thereof and at least one vitamin B3 or derivative thereof are found to synergistically improve wound contraction and cell proliferation, and also reduce inflammation associated with melanin production, increase sGAG release and inhibition of elastase activity, and stimulate barrier formation post aesthetic procedure. Clinical improvements in dyschromia and redness were surprisingly sustained for up to 2 months.

Compositions according to the disclosure comprise at least one C-glycoside or derivative thereof and at least one vitamin B3 or derivative thereof, and methods according to the disclosure combine the use of at least one C-glycoside or derivative thereof and/or at least one vitamin B3 or derivative thereof with treatment of skin using a skin modification stimulus.

In various embodiments, the disclosure relates to the use of at least one C-glycoside or derivative thereof and/or at least one vitamin B3 or derivative thereof in conjunction with various aesthetic procedures for preventing, reducing, and/or improving the appearance of skin. In some embodiments, the disclosure relates to methods comprising applying at least one C-glycoside or derivative thereof and at least one vitamin B3 or derivative thereof, simultaneously or separately, before, during, and/or after a skin procedure such as a skin resurfacing or modification treatment.

In various embodiments, the disclosure relates to methods for treating skin, including improving the appearance of skin, comprising applying at least one C-glycoside or derivative thereof and at least one vitamin B3 or derivative thereof to skin in combination with treatment with a skin modification stimulus. For example, in some embodiments, the disclosure relates to methods for improving the appearance of skin, the methods comprising:
(a) treating the skin with at least one skin modification stimulus,
(b)(1) applying at least one C-glycoside or derivative thereof to the skin, and
(b)(2) applying at least one vitamin B3 or derivative thereof to the skin.

In other embodiments, the disclosure relates to methods for preventing or reducing the adverse effects from aesthetic skin procedures, such as by improving the outcomes of aesthetic skin procedures, such as skin resurfacing treatments comprising applying at least one C-glycoside or derivative thereof and at least one vitamin B3 or derivative thereof to skin tissue prior to a skin injury, e.g. prior to an aesthetic procedure, in combination with a skin modification stimulus treatment. For example, in some embodiments, the disclosure relates to methods for preventing or reducing skin inflammation, the methods comprising:
(a) treating the skin with at least one skin modification stimulus,
(b)(1) applying at least one C-glycoside or derivative thereof to the skin, and
(b)(2) applying at least one vitamin B3 or derivative thereof to the skin.

In another embodiment, the disclosure relates to methods of treating skin to prevent or reduce the formation of skin discoloration after aesthetic procedures, the methods comprising:
(a) treating the skin with at least one skin modification stimulus,
(b)(1) applying at least one C-glycoside or derivative thereof to the skin, and
(b)(2) applying at least one vitamin B3 or derivative thereof to the skin.

In various embodiments, the at least one C-glycoside or derivative thereof and the at least one vitamin B3 or derivative thereof may be applied to skin tissue during treatment with a skin modification stimulus. In other embodiments, the at least one C-glycoside or derivative thereof and the at least one vitamin B3 or derivative thereof may be applied to the skin tissue before and/or after treatment with a skin modification stimulus, for example within a few seconds, about 1 minute, or about 1 hour before and/or after treating the skin with a skin modification stimulus treatment, to within about 6 hours, about 12 hours, about 18 hours, or about 24 hours before and/or after treating the skin with a skin modification procedure. In various embodiments, the at least one C-glycoside or derivative thereof and the at least one vitamin B3 or derivative thereof may be applied to the skin tissue one or more times per day, for example 2 times per day, 3 times per day, or more. In further embodiments, the at least one C-glycoside or derivative thereof and the at least one vitamin B3 or derivative thereof can be applied to the skin tissue at least once a day over a period of one or more consecutive days, for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 47, 48, 49, 50, 51, 52, 53, 54, 55, 56, 57, 58, 59, 60 days, or more, or at least once a day over a period of one or more non-consecutive days, for example every other day, about three times a week, about twice a week, about once a week, about once every 2 weeks, about once every 3 weeks, about once every 4 weeks, or more. In further embodiments, methods according to the disclosure may be repeated one or more times, for example every day, every other day, about three times a week, about twice a week, about once a week, about once every 2 weeks, about once every 3 weeks, about once every 4 weeks, or more with an at-home or clinical-grade skin modification treatment.

In still further embodiments, the methods comprise:
(a) treating skin tissue with at least one type of skin modification stimulus, for example a laser, radiofrequency, an electrical, heat, a low-level light, a needle, or an abrasive instrument; and
(b)(1) applying a composition comprising C13-D-xylopyranoside-2-hydroxypropane to the modified skin tissue within about 6 hours after step (a), wherein the C13-D-xylopyranoside-2-hydroxypropane is present in the composition in an amount ranging from about 0.01% to about 50%, from about 0.025% to about 40%, from about 0.05% to about 30%, from about 0.1% to about 20%, from about 0.2% to about 15%, from about 0.3% to about 12%, from about 0.4% to about 11%, from about 0.5% to about 10%, from about 0.6% to about 9%, from about 0.7% to about 8%, from about 0.8% to about 7%, from about 0.9% to about 6%, from about 1% to about 5%, or from about 2% to about 4% relative to the total weight of the composition, and
(b)(2) applying a composition comprising niacinamide to the modified skin tissue within about 6 hours after step (a), wherein the niacinamide is present in the composition in an amount ranging from about 0.0001% to about 50%, from about 0.0005% to about 45%, from about 0.001% to about 40%, from about 0.0025% to about 35%, from about 0.005% to about 30%, from about 0.0075% to about 25%, from about 0.01% to about 20%, from about 0.025% to about 15%, from about 0.05% to about 10%, from about 0.075% to about 8%, from about 0.1% to about 7%, from about 0.25% to about 6%, from about 0.5% to about 5%, or from about 1% to about 4% by weight, such as from about 1% to about 19%, from about 2% to about 18%, from about 3% to about 17%, such as from about 4% to about 16%, from about 5% to about 15%, from about 6% to about 14%, from about 7% to about 13%, from about 8% to about 12%, or from about 9% to about 11%, by weight, relative to the total weight of the composition, wherein compositions (b)(1) and (b)(2) are applied preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 47, 48, 49, 50, 51, 52, 53, 54, 55, 56, 57, 58, 59, 60 times after treating the skin tissue in step (a), wherein composition (b)(1) and (b)(2) are applied the at least once a day, wherein all amounts are by weight, relative to the total weight of the composition in which it is present.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Implementation of the present technology will now be described, by way of example only, with reference to the attached figures.

FIG. 1 shows PEG2 secretion in untreated fibroblast cultures (negative control) and IL-1a stimulated fibroblast cultures (positive control) including cultures further treated with dexamethasone (Dex) (positive control), hydroxypropyl tetrahydropyrantriol (PX), niacinamide (Vit B3), or a combination of hydroxypropyl tetrahydropyrantriol and niacinamide.

FIG. 2 shows relative α-SMA expression in untreated fibroblast cultures (negative control) and fibroblast cultures and in cultures further treated with Transforming Growth Factor-β (TGF-β) (positive control), hydroxypropyl tetrahydropyrantriol (PX), niacinamide (NAM), or a combination of hydroxypropyl tetrahydropyrantriol and niacinamide.

FIG. 3 shows relative Ki67 protein (Antigen KI-67, or MKI67) expression in fibroblast cultures (negative control) and in cultures further treated with hydroxypropyl tetrahydropyrantriol (PX), niacinamide (NAM), or a combination of hydroxypropyl tetrahydropyrantriol and niacinamide.

FIG. 4 shows relative sGAG release in day T-Skin Softskin™ model epidermal tissues before and after laser ablation, and in tissues further treated hydroxypropyl tetrahydropyrantriol (PX), niacinamide (NAM), or a combination of hydroxypropyl tetrahydropyrantriol and niacinamide.

FIG. 5 shows percent elastase activity inhibition in day in a Neutrophil Elastase Inhibition Assay in samples treated with hydroxypropyl tetrahydropyrantriol (PX), niacinamide (NAM), or a combination of hydroxypropyl tetrahydropyrantriol and niacinamide.

FIG. 6 shows hematoxylin and eosin (H&E), filament aggregating protein (filaggrin), and Transglutaminase-1 (TGM-1) stained cross-sections of fresh, defatted and cleaned of blood residue post-abdominoplasty normal human skin samples model epidermal tissues that have (“microneedling” or “MN”) or have not (“untreated skin”) been subjected to microneedling. Some tissues were cultured with 0.0009% hydroxypropyl tetrahydropyrantriol and 0.2% niacinamide. Cross-sections were obtained and stained on day 6.

FIG. 7 shows an expertly graded global wrinkle, fine line, skin dyschromia, skin roughness, and overall skin health skin parameters at day 14 (D14), day 28 (D28), and day 56 (D56) post-laser procedure for a subjects treated with hydroxypropyl tetrahydropyrantriol (proxylane) and niacinamide.

FIG. 8 shows photographs of laser treated skin immediately after the procedure, and 56 days after the procedure with treatment hydroxypropyl tetrahydropyrantriol (proxylane) and niacinamide.

FIG. 9 shows photographs of laser treated skin immediately after the procedure, and 56 days after the procedure with treatment with hydroxypropyl tetrahydropyrantriol (PX) and niacinamide (NAM) or treatment with Aquafor Healing Ointment (bench).

FIG. 10 shows melanin expression in PGE-2 induced Normal Human Epidermal Melanocytes-Dark Pigment (Lot 1981687, Passage 3, ThermoFisher, Waltham, MA) (positive control) and in cultures further treated with Tranexamic Acid (TXA), hydroxypropyl tetrahydropyrantriol (PX), niacinamide (NAM), or a combination of hydroxypropyl tetrahydropyrantriol and niacinamide.

### DETAILED DESCRIPTION

The disclosure relates to compositions and methods for treating skin tissue, such as by increasing, accelerating, and/or improving healthy skin renewal, and/or improving the appearance of skin. The disclosure also relates to compositions and methods for preventing or minimizing the formation of skin inflammation and skin discoloration before, during or after skin injury, for example prior to a laser treatment or microdermabrasion procedure.

The compositions according to various aspects of the disclosure comprise at least one C-glycoside or derivative, for example at least one C-xylopyranoside or derivative, and in at least certain embodiments comprise hydroxypropyl tetrahydropyrantriol with at least one vitamin B3 or derivative thereof, which may, in at least certain embodiments, comprise niacinamide.

The methods according to various embodiments comprise treating skin tissue with at least one skin modification stimulus and applying at least one C-glycoside or derivative thereof, for example a C-xylopyranoside or derivative thereof, e.g. hydroxypropyl tetrahydropyrantriol, and at least one vitamin B3 or derivative thereof, e.g. niacinamide to the skin tissue. In other embodiments, the methods comprise treating uninjured skin with at least one skin modification stimulus and applying at least one C-glycoside or derivative thereof, for example a C-xylopyranoside or derivative thereof, e.g. hydroxypropyl tetrahydropyrantriol, and at least one vitamin B3 or derivative thereof, e.g. niacinamide to the skin, in order to prevent or reduce inflammation and/or the discoloration of skin after treatment with the skin modification treatment.

Without intending to be limited by theory, it is understood that C-glycosides or derivatives thereof when used with vitamin B3 or derivatives thereof may synergistically work together to reduce inflammation and increase skin regeneration after skin modification procedures by reducing PGE₂ expression, by increasing α-SMA and/or Ki-67 expression, by increasing sGAG secretion, and/or by inhibiting elastase activity.

As such, compositions and methods according to the disclosure surprisingly address the problem of inflammation and skin discoloration in a new and previously unknown manner, via a mechanism that is different from that of known methods. By treating skin tissue with a skin modification stimulus and applying at least one C-glycoside or derivative thereof and at least one vitamin B3 or derivative thereof to the targeted area, it encourages proliferation of new normal skin tissue, rather than inflamed and/or discolored tissue.

Surprisingly, it has been found that in some embodiments, the skin treatment and inflammation prevention methods and compositions according to the disclosure impart broader and/or stronger skin inflammation and hyperpigmentation treatment and prevention benefits to resurfaced and otherwise modified skin, with or without enhanced kinetics, as compared to known skin inflammation and hyperpigmentation treatments.

A more detailed description of compositions, methods, and kits for treating skin and skin tissue is provided below.

### I. Compositions

Compositions according to the disclosure comprise at least one C-glycoside or derivative thereof and/or at least one vitamin B3 or derivative thereof. The compositions may further comprise additional components such carriers, active ingredients other than C-glycosides or derivatives thereof and vitamin B3s or derivatives thereof, and additives typically found in skin care compositions.

A C-glycoside is a sugar moiety linked via a carbon-carbon (C—C) bond to a non-sugar moiety (aglycone). In various embodiments, the C-glycoside may be chosen from C13-D-xylopyranoside-n-propan-2-one; Cα-D-xylopyranoside n-propan-2-one; C13-D-xylopyranoside-2-hydroxypropane; Cα-D-xylopyranoside-2-hydroxypropane; 1-(C13-D-fucopyranoside)-propane-2-one; 1-(Cα-D-fucopyranoside)-propan-2-one; 1-(C13-L-fucopyranoside)-propan-2-one; 1-(Cα-L-fucopyranoside)-propane-2-one; 2-one, 1-(C13-D-fucopyranoside)-2-hydroxypropane, 1-(Cα-D-fucopyranoside)-2-hydroxypropane; 1-(C13-L-fucopyranoside)-2-hydroxypropane; 1-(Cα-L-fucopyranoside)-2-hydroxypropane; 1-(C13-D-glucopyranosyl)-2-hydroxylpropane; 1-(Cα-D-glucopyranosyl)-2-hydroxylpropane; 1-(C13-D-galactopyranosyl)-2-hydroxylpropane; 1-(Cα-D-galactopyranosyl)-2-hydroxylpropane-1-(C13-D-fucofuranosyl) propan-2-one; 1-(Cα-D-fucofuranosyl)-propan-2-one; 1-(C13-L-fucofuranosyl)-propan-2-one; 1-(Cα-L-fucofuranosyl)-propan-2-one; C13-D-maltopyranoside-n-propan-2-one-Cα-D-maltopyranoside-n-propan-2-one-C13-D-maltopyranoside-2-hydroxypropane; Cα-D-maltopyranoside-2-hydroxypropane; as well as derivatives thereof or mixtures thereof.

In various embodiments, the at least one C-glycoside is a C-xylopyranoside. In at least some embodiments, the C-glycoside comprises, consists essentially of, or consists of C13-D-xylopyranoside-2-hydroxypropane, Cα-D-xylopyranoside-2-hydroxypropane, or mixtures thereof. In yet further embodiments, the C-glycoside comprises, consists essentially of, or consists of C13-D-xylopyranoside2-hydroxypropane (also known as hydroxypropyl tetrahydropyrantriol or Proxylane). Hydroxypropyl tetrahydropyrantriol has the structure of Formula (I) below:

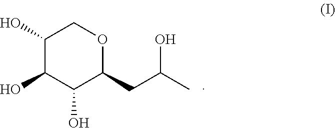

According to various embodiments, compositions according to the disclosure may comprise a total amount of C-glycoside and derivatives thereof from about 0.01% to about 50%, from about 0.025% to about 40%, from about 0.05% to about 30%, from about 0.1% to about 20%, from about 0.2% to about 15%, from about 0.3% to about 12%, from about 0.4% to about 11%, from about 0.5% to about 10%, from about 0.6% to about 9%, from about 0.7% to about 8%, from about 0.8% to about 7%, from about 0.9% to about 6%, from about 1% to about 5%, or from about 2% to about 4% by weight, including all ranges and subranges therebetween using any of the disclosed lower limits as a lower limit and disclosed upper limits as an upper limit, relative to the total weight of the composition in which it is present. For example, the total amount of C-glycoside and derivatives thereof in compositions according to the disclosure may be about 0.01%, about 0.05%, about 0.1%, about 0.25%, about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 5%, about 5.25%, about 5.5%, about 5.75%, about 6%, about 6.25%, about 6.5%, about 6.75%, about 7%, about 7.25%, about 7.5%, about 7.75%, about 8%, about 8.25%, about 8.5%, about 8.75%, about 9%, about 9.25%, about 9.5%, about 9.75%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, about 20%, about 21%, about 22%, about 23%, about 24%, about 25%, about 26%, about 27%, about 28%, about 29%, or about 30% by weight, including all ranges and subranges therebetween using any of the disclosed amounts as a lower limit or upper limit, relative to the total weight of the composition in which it is present.

In various embodiments, the at least one vitamin B3 or derivative thereof is chosen from niacin (nicotinic acid), niacinamide (nicotinamide), nicotinamide riboside, derivatives thereof, or mixtures thereof. In various embodiments, the at least one vitamin B3 is niacinamide.

According to various embodiments, compositions according to the disclosure may comprise a total amount of vitamin B3 and derivatives thereof from about from about 0.0001% to about 50%, from about 0.0005% to about 45%, from about 0.001% to about 40%, from about 0.0025% to about 35%, from about 0.005% to about 30%, from about 0.0075% to about 25%, from about 0.01% to about 20%, from about 0.025% to about 15%, from about 0.05% to about 10%, from about 0.075% to about 8%, from about 0.1% to about 7%, from about 0.25% to about 6%, from about 0.5% to about 5%, or from about 1% to about 4% by weight, such as from about 1% to about 19%, from about 2% to about 18%, from about 3% to about 17%, such as from about 4% to about 16%, from about 5% to about 15%, from about 6% to about 14%, from about 7% to about 13%, from about 8% to about 12%, or from about 9% to about 11% by weight, including all ranges and subranges therebetween using any of the disclosed lower limits as a lower limit and disclosed upper limits as an upper limit, relative to the total weight of the composition in which it is present. For example, the total amount of vitamin B3 and derivatives thereof in compositions according to the disclosure may be about 0.0001%, about 0.0005%, about 0.001%, about 0.0025%, about 0.005%, about 0.0075%, about 0.01%, about 0.025%, about 0.05%, about 0.075%, about 0.1%, about 0.25%, about 0.5%, about 0.75%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, or about 50% by weight, including all ranges and subranges therebetween using any of the disclosed amounts as a lower limit or upper limit, relative to the total weight of the composition in which it is present.

In some embodiments, the compositions according to the disclosure comprise at least one C-glycoside and at least one vitamin B3.

The compositions will generally comprise one or more cosmetically-acceptable carriers, including but not limited to water and/or non-aqueous solvents. Exemplary non-aqueous solvents include glycerin; glycols, such as ethylene glycol, diethylene glycol, triethylene glycol, tetraethylene glycol, pentaethylene glycol, propylene glycol, dipropylene glycol, tripropylene glycol, polyethylene glycol, caprylyl glycol, and hexylene glycol; glycol ethers such as ethylene glycol monomethyl ether, ethylene glycol monoethyl ether, ethylene glycol monobutyl ether, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; and alcohols such as ethanol, propanol, isopropanol, butanol, pentanol, hexanol, heptanol, octanol, decanol, dodecanol, hexadecanol, octadecanol, eicosadecanol, 2-propanol, 2-methyl-1-propanol, 2-methyl-2-butanol, cetyl alcohol, stearyl alcohol, arachidyl alcohol, behenyl alcohol, and cyclohexanol. Mixtures of two or more non-aqueous solvents may also be chosen, and it is contemplated that in various embodiments, the carrier comprises water and at least one non-aqueous solvent.

The compositions may optionally comprise one or more active ingredients other than C-glycosides or derivatives thereof and the vitamin B3 or derivatives thereof, such as, for example, actives that can modulate inflammation (including but not limited to carotenoids, curcumin, steroids, cannabinoids, glycoproteins, essential oils, flavonoids & flavonoid derivatives, phenolic acids, ascorbic acid, polyphenols, marine and algal extracts), actives that can enhance skin barrier (including but not limited to pantothenic acid, ceramides, pseudo ceramides such as 2-Oleyl-1,3-octadecanediol, niacinamides, niacinamide derivatives, carob seed extracts, oligo-galactomannan, colloidal oatmeal, hyaluronic acids, probiotics and β-glucan), and/or actives that can modulate skin fibrosis (including but not limited to inhibitors of TNF, inhibitors of TGF-β, inhibitors of mTOR pathway and kynurenic acid and its derivatives).

The compositions may optionally contain one or more additives such as those typically found in the skin care compositions, including but not limited to oils, waxes, or other fatty substances; gelling agents and/or thickeners; emulsifiers; moisturizing agents; emollients; sunscreens; hydrophilic or lipophilic active agents, such as ceramides; agents for combatting free radicals; bactericides; sequestering agents; preservatives; pH adjusters; skin penetration enhancers; fragrances; surfactants; fillers; natural products or extracts thereof, such as aloe or green tea extract; vitamins; and/or coloring materials. The amount of such additives, if present, will vary depending on the intended use and/or properties of the composition and the effect desired, but will, in individual or combined amount, typically range from about 0.0001% to about 20%, such as from about 0.001% to about 15%, from about 0.01% to about 10%, from about 0.1% to about 7.5%, from about 0.5% to about 5%, or from about 1% to about 3% by weight, relative to the total weight of the composition.

Additives may also include, but are not limited to, panthenol (Vitamin B5), carob seed extract, ginger root extract, polysaccharides (including but not limited to galactomannans, pullulan, beta glucan, alpha glucan, Caesalpinia sphinosa gum), oligosaccharides (including but not limited to oligo-galactomannans), wagonin, baicalin, ceramides/sphingolipids and its derivatives, resveratrol and its derivatives, niacinamide and its derivatives, TXA, pomegranate extract, linoleic acid, linolenic acid, olic acid, vitamin C, vitamin E/tocopherols and its derivatives, polyphenols, flavanols, dihydroflavanols, ellagic acid, flavonoids, collagen and its derivatives, Arnica montana extract, carotenoids and its derivatives, triglycerides, soybean oil, oleuropeins and its derivatives, phospholipids, beta-carotene, micro-algae and algae extracts (including but not limited to Chlorella vulgaris extract, Dunaliella salina extract), pre/pro/post-biotics (including but not limited to Lactococcus ferment lysate, lactobacillus ferment), linseed extract, proanthocyanins, anthocyanins, hydrocyacetophenone, lecithin, GHK-Cu peptide, tripeptide-1, copper peptide, GHK-Mn, Acetyl Tetrapeptides, Tripeptide 10 Citrulline, Palmitoyl Hexapeptide-12, Palmitoyl Tripeptide-1, Lipospondin, Gexapeptide 11, PKEK, GEKG, SA1-11, Tripeptide-3, Pentapeptide-18, Pentapeptide-3, Acetyl Octapeptide-1,3, antimicrobial peptides (including but not limited to and β defensins, cathelicindin, dermcidin), NMF derived peptides (including but not limited to histidine dipeptides, Urocanic acid, pyrrolidone carboxylic acid), Pal-KTTKS and other acyl pentapeptides, KEK and PKEK peptides, Palmitoyl Tetrapeptide-7, Palmitoyl Pentapeptide-4, Tropaeolum Majus Extract, glycoproteins, hyaluronic acid and its derivatives, sh-Polypeptide-5, sh-Polypeptide-11, sh-Oligopeptide-2, sh-polypeptide-6, wheat germ oil, octacosanol, wild yam root extract, bakuchiol, borage oil, geranium oil, protium heptaphyllum resin, hamamelis virginiana extract, citrus medica limonum oil, glutathione, Co-enzyme Q10, disodium acetyl glucosamine phosphate, fermented red ginseng extract, dipeptide diaminobutyroyl benzylamide diacetate, angelica polymorpha sinensis root extract, sericin, superoxide dismutase.

The form of the compositions according to the disclosure is not limited, and may include, for example, liquids, emulsions, suspensions, lotions, creams, gels (including hydrogels), balms, pastes, serums, salves, foams, or the like.

The compositions are typically applied topically and can, in various embodiments, be applied by the consumer as an at-home formulation, or by a professional, e.g. in a clinical setting as part of an aesthetic procedure.

A non-limiting example of a composition according to the disclosure includes a carrier, for example water and/or at least one non-aqueous solvent, optionally at least one active ingredient other than C-glycosides or derivatives thereof, and/or at least one additive, and a total amount of C-glycosides and derivatives thereof ranging from about 0.01% to about 50%, from about 0.025% to about 40%, from about 0.05% to about 30%, from about 0.1% to about 20%, from about 0.2% to about 15%, from about 0.3% to about 12%, from about 0.4% to about 11%, from about 0.5% to about 10%, from about 0.6% to about 9%, from about 0.7% to about 8%, from about 0.8% to about 7%, from about 0.9% to about 6%, from about 1% to about 5%, or from about 2% to about 4% by weight, including all ranges and subranges therebetween, relative to the total weight of the composition in which it is present. A further non-limiting example of a composition according to the disclosure includes a carrier comprising water and optionally at least one non-aqueous solvent, optionally at least one active ingredient other than C-glycosides or derivatives thereof and/or at least one additive, and hydroxypropyl tetrahydropyrantriol present in an amount ranging from about 0.01% to about 50%, from about 0.025% to about 40%, from about 0.05% to about 30%, from about 0.1% to about 20%, from about 0.2% to about 15%, from about 0.3% to about 12%, from about 0.4% to about 11%, from about 0.5% to about 10%, from about 0.6% to about 9%, from about 0.7% to about 8%, from about 0.8% to about 7%, from about 0.9% to about 6%, from about 1% to about 5%, or from about 2% to about 4% by weight, including all ranges and subranges therebetween, relative to the total weight of the composition in which it is present.

Another non-limiting example of a composition according to the disclosure includes a carrier, for example water and/or at least one non-aqueous solvent, optionally at least one active ingredient other than vitamin B3 or derivatives thereof, and/or at least one additive, and a total amount of vitamin B3 and derivatives thereof ranging from about 0.0001% to about 50%, from about 0.0005% to about 45%, from about 0.001% to about 40%, from about 0.0025% to about 35%, from about 0.005% to about 30%, from about 0.0075% to about 25%, from about 0.01% to about 20%, from about 0.025% to about 15%, from about 0.05% to about 10%, from about 0.075% to about 8%, from about 0.1% to about 7%, from about 0.25% to about 6%, from about 0.5% to about 5%, or from about 1% to about 4% by weight, such as from about 1% to about 19%, from about 2% to about 18%, from about 3% to about 17%, such as from about 4% to about 16%, from about 5% to about 15%, from about 6% to about 14%, from about 7% to about 13%, from about 8% to about 12%, or from about 9% to about 11% by weight, including all ranges and subranges therebetween, relative to the total weight of the composition in which it is present. A further non-limiting example of a composition according to the disclosure includes a carrier comprising water and optionally at least one non-aqueous solvent, optionally at least one active ingredient other than niacinamide thereof and/or at least one additive, and niacinamide present in an amount ranging from about 0.0001% to about 50%, from about 0.0005% to about 45%, from about 0.001% to about 40%, from about 0.0025% to about 35%, from about 0.005% to about 30%, from about 0.0075% to about 25%, from about 0.01% to about 20%, from about 0.025% to about 15%, from about 0.05% to about 10%, from about 0.075% to about 8%, from about 0.1% to about 7%, from about 0.25% to about 6%, from about 0.5% to about 5%, or from about 1% to about 4% by weight, such as from about 1% to about 19%, from about 2% to about 18%, from about 3% to about 17%, such as from about 4% to about 16%, from about 5% to about 15%, from about 6% to about 14%, from about 7% to about 13%, from about 8% to about 12%, or from about 9% to about 11% by weight, including all ranges and subranges therebetween, relative to the total weight of the composition in which it is present.

In another embodiment, a composition according to the disclosure includes a carrier, for example water and/or at least one non-aqueous solvent, optionally at least one active ingredient other than C-glycosides or derivatives thereof and vitamin B3 or derivatives thereof, and/or at least one additive, a total amount of C-glycosides and derivatives thereof ranging from about 0.01% to about 50%, from about 0.025% to about 40%, from about 0.05% to about 30%, from about 0.1% to about 20%, from about 0.2% to about 15%, from about 0.3% to about 12%, from about 0.4% to about 11%, from about 0.5% to about 10%, from about 0.6% to about 9%, from about 0.7% to about 8%, from about 0.8% to about 7%, from about 0.9% to about 6%, from about 1% to about 5%, or from about 2% to about 4% by weight, and a total amount of vitamin B3 and derivatives thereof ranging from about 0.0001% to about 50%, from about 0.0005% to about 45%, from about 0.001% to about 40%, from about 0.0025% to about 35%, from about 0.005% to about 30%, from about 0.0075% to about 25%, from about 0.01% to about 20%, from about 0.025% to about 15%, from about 0.05% to about 10%, from about 0.075% to about 8%, from about 0.1% to about 7%, from about 0.25% to about 6%, from about 0.5% to about 5%, or from about 1% to about 4% by weight, such as from about 1% to about 19%, from about 2% to about 18%, from about 3% to about 17%, such as from about 4% to about 16%, from about 5% to about 15%, from about 6% to about 14%, from about 7% to about 13%, from about 8% to about 12%, or from about 9% to about 11% by weight, including all ranges and subranges therebetween, relative to the total weight of the composition in which it is present. The weight ratio of the total amount the C-glycoside(s) and derivatives thereof to the total amount of the vitamin B3 and derivatives thereof applied to the skin may range from about 0.001:1 to about 10:1, about 0.005:1 to about 8:1, about 0.01:1 to about 6:1, about 0.05:1 to about 4:1, about 0.1:1 to about 2:1, or about 0.5:1 to about 1.5:1.

### II. Methods

In various embodiments, the disclosure relates to methods of treating skin tissue to improve the appearance of skin. In further embodiments, the disclosure relates to methods of reducing skin inflammation after a skin modification treatment. In yet further embodiments, the disclosure relates to methods reducing the secretion of prostaglandin E2 in modified skin tissue, reducing the secretion of alpha smooth muscle actin in modified skin tissue, increasing cell proliferation in modified skin tissue, reducing the secretion of melanin in modified skin tissue, and/or increasing the secretion of at least one sulfated glycosaminoglycan in modified skin tissue.

Typically, the methods comprise applying at least one C-glycoside or derivative thereof, for example a C-xylopyranoside or derivative thereof, e.g. niacinamide, to a desired area to be treated (referred to collectively in some instances herein as a “target area”), before, during, and/or after treating the skin with a stimulus for modifying skin (referred to interchangeably herein as a “skin modification stimulus”). In various methods, the C-glycoside or derivative thereof comprises, consists essentially of, or consists of C-xylopyranosides and/or derivatives thereof, and in further methods, the C-glycoside or derivative thereof comprises, consists essentially of, or consists of hydroxypropyl tetrahydropyrantriol. In various methods, the vitamin B3 or derivative thereof comprises, consists essentially of, or consists of niacinamide (nicotinamide).

In methods according to the disclosure, useful skin modification stimuli include those used in the skin care field to modify or damage skin in order to encourage new growth, generally in a controlled manner. These treatments typically damage the outermost layers of the skin tissue to a relatively mild degree, which sets into motion the body's natural healing process. Such treatments may also stimulate the production of collagen. Thus, any known method or device for modifying or damaging the skin tissue can be used in the methods according to the disclosure. For example, in various embodiments, the skin modification stimulus may modify or damage the skin tissue by cutting, burning, chemically damaging, ablating, micro-ablating, coagulating, or otherwise affecting the corneal layer and subcutaneous tissue of the target area. Therefore, as used herein, “modified skin tissue” is skin tissue that has been modified or damaged with one or more skin modification stimuli, and is healing from treatment with the skin modification stimulus.

The skin modification stimulus used in the methods according to the disclosure may, in various embodiments, be invasive or non-invasive. By way of non-limiting example, the skin modification stimulus can be a laser, plasma, radiofrequency, a high-intensity focused ultrasound, an electrical field, heat, a low-level light device, a needle, a microneedle, an abrasive element, a chemical agent such as a chemical exfoliating agent, or the like, and the skin modification stimulus treatment may be a laser procedure, a microneedling procedure, a high-intensity focused ultrasound procedure, an electroporation procedure, a dermabrasion procedure, a microdermabrasion procedure, a plasma skin rejuvenation procedure, a chemical exfoliation procedure, or the like.

Any variety of cosmetic lasers, distinguished by their wavelength and their mode of delivering the laser, can be used. In various embodiments, the target area may be treated with ablative or non-ablative lasers. Ablative lasers may include, e.g., carbon dioxide or erbium lasers. Non-ablative lasers may include, e.g., pulsed light, pulsed-dye, and fractional lasers. Lasers can also be modified when combined with other elements, such as gases, precious stones, and metals. Nonlimiting examples include Clear+Brilliant® lasers, fractional photothermolysis lasers, alexandrite lasers, carbon dioxide (CO₂) lasers, erbium (Er:YAG) lasers, intense pulsed light (IPL) lasers, Nd:YAG lasers, pulse dye lasers, Q-switched lasers, picosecond lasers, etc.

In various embodiments, the target area may be treated with mechanical microneedling, which uses a motorized pen with tiny slender needles to create thousands of channels that are up to 2.5 millimeters deep. These channels disrupt the uninjured skin tissue to allow for growth of new skin. Non-limiting examples of mechanical microneedling devices that can be used include SkinPen Precision, MDpen, Skin Stylus, etc.

In some embodiments, the target area may be treated with fractional radiofrequency (RF) microneedling, which is a heat-based therapy that uses thicker needles to transmit heat deep into the skin. RF microneedling thermally coagulates tissue to stimulate collagen denaturation and renewal. Radiofrequency (RF) microneedling generates less microchannels than mechanical needling. In an embodiment, the target area may be treated with standard or short-pulse RF microneedling. Non-limiting examples of RF devices that may be used include Scarlet SRF, Agnes RF, Endymed Intensif, Secret RF, Vivace, Virtue RF, Morpheus8, etc.

The target area can also be treated with ultrasound, such as through ultratherapy. Ultrasound can thermally loosen, stretch, and re-orient collagen tissue. Ultrasound mechanically acts on skin tissue as a micro-massage and produces microscopic droplets of oxygen (calvitation) from the vibration.

Electroporation mesotherapy can also be utilized with the disclosed methods. Electroporation uses electricity to boost the permeability of the skin tissue membrane by creating microscopic channels that open up pathways deep into the skin, thus disrupting skin tissue.

Dermabrasion, including microdermabrasion, may also be used to treat the target area in some embodiments. Dermabrasion involves the controlled deeper abrasion of the upper to mid layers of the uninjured skin tissue with any variety of strong abrasive devices such as a wire brush, diamond wheel (or fraise), or serrated wheel. Exemplary embodiments may include crystal microdermabrasion or diamond microdermabrasion.

In other embodiments, plasma skin rejuvenation may be used in the disclosed methods. Plasma skin regeneration can be used to deliver energy in the form of plasma to the target area, resembling a thermal device and a dry peel.

In certain embodiments, a chemical agent such as a chemical exfoliating or peeling agent (also known as chemexfoliation or derma-peeling) can be used as a skin modification stimulus. Exemplary and non-limiting chemical agents that may be used to treat the target area include chemical agents capable of acting either directly on peeling by encouraging exfoliation, such as β-hydroxyacids (BHAs), for example salicylic acid and derivatives thereof (including n-octanoyl 5-salicylic acid, otherwise known as capryloyl salicylic acid (INCI name)); α-hydroxyacids (AHAs), such as glycolic, lactic, tartric, malic, or mandelic acids; 8-hexadecene-1,16-dicarboxylic acid, or 9-octadecene dioic acid; urea and derivatives thereof; trichloroacetic acid; gentisic acid and derivatives thereof; oligofuctoses; cinnamic acid; extract of Saphora japonica; resveratrol; resorcinol; carbolic acid (phenol); or mixtures thereof.

Further exemplary and non-limiting chemical peeling agents that may be used include compounds involved in peeling or degrading corneodesmosomes, such as aminosulfonic compounds, e.g. 4-(2-hydroxyethyl) piperazine-1-propanesulfonic acid (HEPES); 2-oxothiazolidine-4-carboxylic acid (procysteine) and derivatives thereof; the derivatives of α-amino acids of the glycine type (as described in EP-0 852 949) and also sodium methyl glycine diacetate sold by BASF under the trade name Trilon M; honey; derivatives of sugar such as O-octanoyl-6-D-maltose and N-acetyl glucosamine; or mixtures thereof.

As still further exemplary and non-limiting chemical peeling agents suitable for use in compositions according to the disclosure, mention be made of EDTA and derivatives thereof, laminaria extracts, O-linoleyl-6-D-gluocose; (3-hydroxy-2-pentylcyclopentyl) acetic acid, glycerol trilactate, S carboxymethyl cysteine, silica-containing derivatives of salicylate such as those described in patent EP 0 796 861, oligofuctases such as those described in patent EP 0 218 200, agents having effects on transglutaminase as in patent EP 0 899 330; extract of the flower ficus opuntia indica such as Exfolactive® from Silab; 8-hexadecene 1,16-dicarboxylic acid; esters of glucose and of vitamin F; or mixtures thereof.

The chemical agent(s) may, in certain embodiments, be chosen from α-hydroxy acids such as citric, lactic, glycolic, malic, tartric, or mandelic acids; β-hydroxy acids such as salicylic acid or derivatives thereof, e.g. n-octanoyl-5-salicylic acid; tricholoacetic acid; phenols; croton oil peels; or mixtures thereof.

The C-glycoside or derivative thereof will typically remain on the skin, which may or may not be modified skin tissue for a time sufficient to achieve the desired benefits. Thus, it may be preferable to leave the C-glycoside or derivative thereof on the skin until it penetrates therethrough or is removed in the ordinary course of personal hygiene, e.g. The next time the skin is washed. For example, in some embodiments, the at least one C-glycoside or derivative thereof is left on the skin for a desired period of time, for example at least 10 seconds, at least 20 seconds, at least 30 seconds, at least 40 seconds, at least 50 seconds, at least 1 minute, at least 2 minutes, at least 3 minutes, at least 5 minutes, at least 10 minutes, at least 15 minutes, at least 20 minutes, at least 25 minutes, at least 30 minutes, at least 35 minutes, at least 40 minutes, at least 45 minutes, at least 1 hour, at least 2 hours, at least 5 hours, at least 8 hours, at least 10 hours, at least 15 hours, at least 20 hours, at least 24 hours, at least 36 hours, or at least 48 hours before it is removed, such as by, for example, wiping or rinsing the skin. For example, the at least one C-glycoside or derivative thereof may be left on the skin for a period of time ranging from 5 minutes to 60 minutes, such as from 5 minutes to 50 minutes, from 5 minutes to 40 minutes, from 5 minutes to 35 minutes, from 5 minutes to 30 minutes, from 5 minutes to 25 minutes, from 5 minutes to 20 minutes, from 5 minutes to 15 minutes, or from 5 minutes to 10 minutes. As a further example, the at least one C-glycoside or derivative thereof may be left on the skin for a period of time ranging from 10 minutes to 60 minutes, such as from 10 minutes to 50 minutes, from 10 minutes to 40 minutes, from 10 minutes to 35 minutes, from 10 minutes to 30 minutes, from 10 minutes to 25 minutes, from 10 minutes to 20 minutes, or from 10 minutes to 15 minutes.

The vitamin B3 or derivative thereof will also typically remain on the skin, which may or may not be modified skin tissue for a time sufficient to achieve the desired benefits. Thus, it may be preferable to leave the vitamin B3 or derivative thereof on the skin until it penetrates therethrough or is removed in the ordinary course of personal hygiene, e.g. The next time the skin is washed. For example, in some embodiments, the at least one vitamin B3 or derivative thereof is left on the skin for a desired period of time, for example at least 10 seconds, at least 20 seconds, at least 30 seconds, at least 40 seconds, at least 50 seconds, at least 1 minute, at least 2 minutes, at least 3 minutes, at least 5 minutes, at least 10 minutes, at least 15 minutes, at least 20 minutes, at least 25 minutes, at least 30 minutes, at least 35 minutes, at least 40 minutes, at least 45 minutes, at least 1 hour, at least 2 hours, at least 5 hours, at least 8 hours, at least 10 hours, at least 15 hours, at least 20 hours, at least 24 hours, at least 36 hours, or at least 48 hours before it is removed, such as by, for example, wiping or rinsing the skin. For example, the at least one vitamin B3 or derivative thereof may be left on the skin for a period of time ranging from 5 minutes to 60 minutes, such as from 5 minutes to 50 minutes, from 5 minutes to 40 minutes, from 5 minutes to 35 minutes, from 5 minutes to 30 minutes, from 5 minutes to 25 minutes, from 5 minutes to 20 minutes, from 5 minutes to 15 minutes, or from 5 minutes to 10 minutes. As a further example, the at least one vitamin B3 or derivative thereof may be left on the skin for a period of time ranging from 10 minutes to 60 minutes, such as from 10 minutes to 50 minutes, from 10 minutes to 40 minutes, from 10 minutes to 35 minutes, from 10 minutes to 30 minutes, from 10 minutes to 25 minutes, from 10 minutes to 20 minutes, or from 10 minutes to 15 minutes.

The steps of applying a C-glycoside or derivative thereof and applying a vitamin B3 or derivative thereof may occur separately or simultaneously.

The step of applying a C-glycoside or derivative thereof may comprise one or more applications, such as two or more applications, three or more applications, etc., to the target area. Thus, in various embodiments, the methods comprise applying at least one C-glycoside or derivative thereof to the target area at least one time before, at least one time during, and/or at least one time after treatment with the skin modification stimulus. For example, the at least one C-glycoside or derivative thereof may be applied to the target area at least one time before, at least one time during, and at least one time after treatment with the skin modification stimulus treatment. As another example, the methods comprise applying at least one C-glycoside or derivative thereof to the target area at least two times before, at least two times during, and/or at least two times after treatment with the skin modification stimulus. As yet a further example, the methods comprise applying at least one C-glycoside or derivative thereof to the target area at least one time, such as at least two times, before treatment with the skin modification stimulus, and at least one time, such as at least two times, after treatment with the skin modification stimulus. As a still further example, the methods comprise applying at least one C-glycoside or derivative thereof to the target area at least one time, such as at least two times, after treatment with the skin modification stimulus.

The step of applying a vitamin B3 or derivative thereof may also comprise one or more applications, such as two or more applications, three or more applications, etc., to the target area. Thus, in various embodiments, the methods comprise applying at least one vitamin B3 or derivative thereof to the target area at least one time before, at least one time during, and/or at least one time after treatment with the skin modification stimulus. For example, the at least one vitamin B3 or derivative thereof may be applied to the target area at least one time before, at least one time during, and at least one time after treatment with the skin modification stimulus treatment. As another example, the methods comprise applying at least one vitamin B3 or derivative thereof to the target area at least two times before, at least two times during, and/or at least two times after treatment with the skin modification stimulus. As yet a further example, the methods comprise applying at least one vitamin B3 or derivative thereof to the target area at least one time, such as at least two times, before treatment with the skin modification stimulus, and at least one time, such as at least two times, after treatment with the skin modification stimulus. As a still further example, the methods comprise applying at least one vitamin B3 or derivative thereof to the target area at least one time, such as at least two times, after treatment with the skin modification stimulus.

In various embodiments, the at least one C-glycoside or derivative thereof can be applied to the target area at least one time per day, at least two times per day, or at least three times per day, such as from one to five times a day, from one to four times a day, from one to three times a day, or from one to two times a day, for example once per day, before and/or after treatment with the skin modification stimulus. The at least one C-glycoside or derivative thereof can be applied to the target area at least one time per day, for example at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 consecutive or non-consecutive days, before and/or after treatment with the skin modification stimulus. The at least one C-glycoside or derivative thereof may be applied to the target area at least one time per day, for example at least about 1, about 2, about 3, about 4, about 5, about 6, about 7 days per week. For example, the at least one C-glycoside or derivative thereof may be applied to the target area at least two times per day, wherein the second or subsequent application is at least about 6 hours, for example at least about 8 hours, at least about 10 hours, at least about 12 hours, or at least about 15 hours after the prior application.

Similarly, the at least one vitamin B3 or derivative thereof can be applied to the target area at least one time per day, at least two times per day, or at least three times per day, such as from one to five times a day, from one to four times a day, from one to three times a day, or from one to two times a day, for example once per day, before and/or after treatment with the skin modification stimulus. The at least one vitamin B3 or derivative thereof can be applied to the target area at least one time per day, for example at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 consecutive or non-consecutive days, before and/or after treatment with the skin modification stimulus. The at least one vitamin B3 or derivative thereof may be applied to the target area at least one time per day, for example at least about 1, about 2, about 3, about 4, about 5, about 6, about 7 days per week. For example, the at least one vitamin B3 or derivative thereof may be applied to the target area at least two times per day, wherein the second or subsequent application is at least about 6 hours, for example at least about 8 hours, at least about 10 hours, at least about 12 hours, or at least about 15 hours after the prior application.

In various embodiments, the methods according to the disclosure may be performed as described herein for at least one month or more, for example for 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 47, 48, 49, 50, 51, 52, 53, 54, 55, 56, 57, 58, 59, or 60 consecutive or non-consecutive months, or for any period of time using any of the foregoing as upper and lower limits to such time period range.

In various embodiments, the at least one C-glycoside or derivative thereof is applied to the target area within about 1 minute, such as within about 5 minutes, within about 10 minutes, within about 15 minutes, within about 20 minutes, within about 30 minutes, within about 45 minutes, within about 60 minutes, within about 90 minutes, within about 2 hours, within about 3 hours, within about 4 hours, within about 5 hours, within about 6 hours, within about 8 hours, within about 10 hours, within about 12 hours, within about 18 hours, within about 24 hours, within about 36 hours, or within about 48 hours before and/or after treating the skin tissue with a skin modification stimulus treatment. In further embodiments, the at least one vitamin B3 or derivative thereof is applied to the target area within about 1 minute, such as within about 5 minutes, within about 10 minutes, within about 15 minutes, within about 20 minutes, within about 30 minutes, within about 45 minutes, within about 60 minutes, within about 90 minutes, within about 2 hours, within about 3 hours, within about 4 hours, within about 5 hours, within about 6 hours, within about 8 hours, within about 10 hours, within about 12 hours, within about 18 hours, within about 24 hours, within about 36 hours, or within about 48 hours before and/or after treating the skin tissue with a skin modification stimulus treatment.

In various methods according to the disclosure, applying at least one C-glycoside or derivative thereof is accomplished by applying a composition comprising at least one C-glycoside or derivative thereof, for example a composition comprising at least one C-xylopyranoside or derivative thereof, e.g. hydroxypropyl tetrahydropyrantriol, to the target area before, during, and/or after the skin tissue is treated with a skin modification stimulus. In further methods according to the disclosure, applying at least one vitamin B3 or derivative thereof is accomplished by applying a composition comprising at least one vitamin B3 or derivative thereof, for example a composition comprising niacinamide to the target area before, during, and/or after the skin tissue is treated with a skin modification stimulus. In an embodiment, the at least one C-glycoside or derivative thereof and the at least one vitamin B3 are combined in one composition and applied simultaneously using methods of the disclosure.

In at least some embodiments, treatments with the skin modification stimulus are carried out with at-home devices. As non-limiting examples, microneedling and microdermabrasion can be accomplished with at-home devices. Such at-home devices can be sold in kits with compositions comprising at least one C-glycoside or derivative thereof, at least one vitamin B3 or derivative thereof, or combinations thereof, for example a first composition comprising at least one C-xylopyranoside or derivative thereof, e.g. hydroxypropyl tetrahydropyrantriol, and a second composition comprising a vitamin B3 or derivative thereof, e.g. niacinamide. Such at-home devices can be sold in kits with a composition comprising at least one C-glycoside or derivative thereof and at least one vitamin B3 or derivative thereof.

The methods according to the disclosure treat skin using the skin modification stimuli according to known, typical procedures. Thus, although it is possible to do so, it is not necessary to vary the length, intensity, or other parameters of treatments with the skin modification stimuli from those which are currently performed in order to obtain the surprising and unexpected inflammation reducing benefits and/or improvement or reduction in skin discoloration according to the disclosure. For example, in a typical microdermabrasion procedure, a device having exfoliating crystals is moved across the skin with light pressure for a period of about 30 minutes. In a method according to the disclosure where an abrasive element is chosen as a skin modification stimulus, the same microdermabrasion procedure can be performed and the at least one C-glycoside or derivative thereof and the at least one vitamin B3 can be applied to the target area before, during, and/or after the procedure.

According to various embodiments of the disclosure, the methods comprise treating skin tissue by applying at least one C-glycoside or derivative thereof and at least one niacinamide to the skin tissue in connection with treating the skin tissue with a skin modification stimulus. In various embodiments, the methods comprise:
(a) treating skin tissue with at least one skin modification stimulus,
(b)(1) applying at least one C-glycoside or derivative thereof to the skin tissue, and
(b)(2) applying at least one vitamin B3 or derivative thereof to the skin tissue.

In some embodiments, the step of (a) treating skin tissue with at least one skin modification stimulus occurs before the steps of (b)(1) applying at least one C-glycoside or derivative thereof to the skin tissue, such that the at least one C-glycoside or derivative thereof is applied to modified skin tissue, and (b)(2) applying at least one vitamin B3 or derivative thereof to the skin tissue, such that the at least one vitamin B3 or derivative thereof is applied to modified skin tissue. In some embodiments, the step of (b)(1) applying at least one C-glycoside or derivative thereof to the skin tissue comprises applying a composition comprising at least one C-glycoside or derivative thereof and at least one carrier to the skin tissue before and/or after step (a), wherein the total amount of C-glycosides and derivatives thereof present in the composition ranges from about 0.01% to about 50%, from about 0.025% to about 40%, from about 0.05% to about 30%, from about 0.1% to about 20%, from about 0.2% to about 15%, from about 0.3% to about 12%, from about 0.4% to about 11%, from about 0.5% to about 10%, from about 0.6% to about 9%, from about 0.7% to about 8%, from about 0.8% to about 7%, from about 0.9% to about 6%, from about 1% to about 5%, or from about 2% to about 4% by weight, relative to the total weight of the composition. In some embodiments, the step of (b)(2) applying at least one vitamin B3 or derivative thereof to the skin tissue comprises applying a composition comprising at least one vitamin B3 or derivative thereof and at least one carrier to the skin tissue before and/or after step (a), wherein the total amount of vitamin B3 and derivatives thereof present in the composition ranges from about 0.0001% to about 50%, from about 0.0005% to about 45%, from about 0.001% to about 40%, from about 0.0025% to about 35%, from about 0.005% to about 30%, from about 0.0075% to about 25%, from about 0.01% to about 20%, from about 0.025% to about 15%, from about 0.05% to about 10%, from about 0.075% to about 8%, from about 0.1% to about 7%, from about 0.25% to about 6%, from about 0.5% to about 5%, or from about 1% to about 4% by weight, such as from about 1% to about 19%, from about 2% to about 18%, from about 3% to about 17%, such as from about 4% to about 16%, from about 5% to about 15%, from about 6% to about 14%, from about 7% to about 13%, from about 8% to about 12%, or from about 9% to about 11% by weight, relative to the total weight of the composition. According to further embodiments of the disclosure, the methods comprise applying at least one C-glycoside or derivative thereof to skin prior to a skin injury and/or applying at least one vitamin B3 or derivative thereof to skin prior to a skin injury, e.g. prior to a surgical procedure in connection with treating the skin with a skin modification stimulus. In various embodiments, the methods comprise:
(a) treating uninjured skin with at least one skin modification stimulus,
(b)(1) applying at least one C-glycoside or derivative thereof to the skin, and
(b)(2) applying at least one vitamin B3 or derivative thereof to the skin.

In some embodiments, the step of (a) treating uninjured skin with at least one skin modification stimulus occurs before the step of (b)(1) applying at least one C-glycoside or derivative thereof to the uninjured skin, such that the at least one C-glycoside or derivative thereof is applied to modified skin tissue and/or the step of (b)(2) applying at least one vitamin B3 or derivative thereof to the uninjured skin, such that the at least one vitamin B3 or derivative thereof is applied to modified skin tissue. In some embodiments, the step of (b)(1) applying at least one C-glycoside or derivative thereof to the uninjured skin comprises applying a composition comprising at least one C-glycoside or derivative thereof and at least one carrier to the uninjured skin before and/or after step (a), wherein the total amount of C-glycosides and derivatives thereof present in the composition ranges from about 0.01% to about 50%, from about 0.025% to about 40%, from about 0.05% to about 30%, from about 0.1% to about 20%, from about 0.2% to about 15%, from about 0.3% to about 12%, from about 0.4% to about 11%, from about 0.5% to about 10%, from about 0.6% to about 9%, from about 0.7% to about 8%, from about 0.8% to about 7%, from about 0.9% to about 6%, from about 1% to about 5%, or from about 2% to about 4% by weight, relative to the total weight of the composition. In some embodiments, the step of (b)(2) applying at least one vitamin B3 or derivative thereof to the uninjured skin comprises applying a composition comprising at least one vitamin B3 or derivative thereof and at least one carrier to the uninjured skin before and/or after step (a), wherein the total amount of vitamin B3 and derivatives thereof present in the composition ranges from about 0.0001% to about 50%, from about 0.0005% to about 45%, from about 0.001% to about 40%, from about 0.0025% to about 35%, from about 0.005% to about 30%, from about 0.0075% to about 25%, from about 0.01% to about 20%, from about 0.025% to about 15%, from about 0.05% to about 10%, from about 0.075% to about 8%, from about 0.1% to about 7%, from about 0.25% to about 6%, from about 0.5% to about 5%, or from about 1% to about 4% by weight, such as from about 1% to about 19%, from about 2% to about 18%, from about 3% to about 17%, such as from about 4% to about 16%, from about 5% to about 15%, from about 6% to about 14%, from about 7% to about 13%, from about 8% to about 12%, or from about 9% to about 11% by weight, relative to the total weight of the composition.

Optionally, in some embodiments, one or more additional active agents (other than C-glycosides or derivatives thereof and vitamin B3 or derivatives thereof) can be applied to the skin either as part of a composition comprising the at least one C-glycoside or derivative thereof and/or vitamin B3 or derivatives thereof or separately, including by way of example actives that can modulate inflammation (including but not limited to carotenoids, curcumin, steroids, cannabinoids, glycoproteins, essential oils, flavonoids & flavonoid derivatives, phenolic acids, ascorbic acid, polyphenols, marine and algal extracts) actives that can enhance skin barrier (including but not limited to pantothenic acid, ceramides, pseudo ceramides such as 2-oleyl-1,3-octadecanediol, niacinamides, niacinamide derivatives, carob seed extracts, oligo-galactomannan, colloidal oatmeal, hyaluronic acids, probiotics and β-glucan), and/or actives that can modulate skin fibrosis (including but not limited to inhibitors of TNF, inhibitors of TGF-β, inhibitors of mTOR pathway and kynurenic acid and its derivatives). The additional active agent(s) may be applied to the target area before, during, and/or after the uninjured skin tissue is treated with a skin modification stimulus, and/or before, during, and/or after the at least one C-glycoside or derivative thereof is applied to the target area.

In some embodiments, at least one additional composition, such as, for example, moisturizer, sunscreen, and/or anti-aging compositions, may be applied to the target area before and/or after application of the at least one C-glycoside or derivative thereof and/or the at least one vitamin B3 or derivative thereof. In various embodiments, the at least one C-glycoside or derivative thereof and/or the at least one vitamin B3 or derivative thereof is applied to the target area first, and is not removed prior to application of at the least one additional skin composition. In another embodiment, the at least one additional skin composition is applied to the target area first, and is not removed prior to application of the at least one C-glycoside or derivative thereof or the at least one vitamin B3 or derivative thereof.

If desired, any of the disclosed steps or methods may be repeated one or more times, using the same or a different skin modification stimulus, and/or the same or a different C-glycoside or derivative and/or vitamin B3 or derivative. If different, treatment with a skin modification stimulus may be different in any manner, for example may use different skin modification stimuli, may use the same stimulus but for a different length of time, etc. Likewise, if different, application of a C-glycoside or derivative may be different in any manner, for example may be different C-glycosides, may be the same C-glycoside but at different concentrations, etc. Similarly, if different, application of a vitamin B3 or derivative may be different in any manner, for example may be different vitamin B3s, may be the same vitamin B3 but at different concentrations, etc.

For example, one method may comprise repeating treatment with the skin modification stimulus one or more times before and/or after the at least one C-glycoside or derivative thereof and/or the at least one vitamin B3 or derivative thereof is applied to the target area, where the treatments with the skin modification stimulus may be the same or different. Another exemplary method may comprise repeating application of at least one C-glycoside or derivative thereof and/or the at least one vitamin B3 or derivative thereof to the target area one or more times before and/or after treatment with the skin modification stimulus, where the C-glycoside or derivative may be the same or different and/or the vitamin B3 or derivative may be the same or different.

As another non-limiting example, a first skin modification stimulus may be a laser, where the first treatment is a Sciton Halo 1470 nm laser procedure, and a second skin modification stimulus may be an abrasive element, where the second treatment is a microdermabrasion procedure. As yet another non-limiting example, a first skin modification stimulus may be a laser, where the first treatment is a Clear+Brilliant® laser procedure, and a second treatment may be a second Clear+Brilliant® laser procedure.

In another example still, a first step of applying at least one C-glycoside or derivative thereof to skin of an individual prior to surgery before, during, and/or after treating the skin with at least one skin modification stimulus; and then, after the individual's surgical wound heals for 7 days, a second step of applying at least one C-glycoside or derivative thereof to skin tissue formed at the site of the wound before, during, and/or after treating the skin tissue with at least one skin modification stimulus, wherein either or both of the C-glycoside or derivative thereof and the skin modification stimulus in the first and second steps are the same or different. Similarly, in another example still, a first step of applying at least one vitamin B3 or derivative thereof to skin of an individual prior to surgery before, during, and/or after treating the skin with at least one skin modification stimulus; and then, after the individual's surgical wound heals for 7 days, a second step of applying at least one vitamin B3 or derivative thereof to skin tissue formed at the site of the wound before, during, and/or after treating the tissue with at least one skin modification stimulus, wherein either or both of the vitamin B3 or derivative thereof and the skin modification stimulus in the first and second steps are the same or different.

As a further non-limiting example, a method may be a method of treating skin tissue in order to improve the appearance of skin thereof, where a first skin modification stimulus is a laser and the first treatment is a Clear+Brilliant® laser procedure, and a second skin modification stimulus is an abrasive element where the second treatment is a microdermabrasion procedure, with application of at least one C-glycoside or derivative thereof to the target area before, between, during, and/or after the first and second treatments, where the C-glycoside or derivative in each application is the same or different, and with application of at least one vitamin B3 or derivative thereof to the target area before, between, during, and/or after the first and second treatments, where the vitamin B3 or derivative in each application is the same or different.

A person skilled in the art will appreciate based on the foregoing that many additional variations in treatment regimens according to the disclosure are contemplated to be within the scope of the disclosure.

In this application, the use of the singular includes the plural unless specifically stated otherwise. Thus, the terms “a,” “an,” and “the” are understood to encompass the plural as well as the singular; thus, “a skin modification stimulus” should be understood to mean “at least one skin modification stimulus” unless expressly stated otherwise. In this application, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one unit unless specifically stated otherwise.

The term “and/or” should be understood to include both the conjunctive and the disjunctive and expressly covers instances of either. For example, applying a C-glycoside or derivative thereof to a target area “before and/or after” treatment with a skin modification stimulus includes applying a C-glycoside or derivative to a target area before treatment with a skin modification stimulus and after treatment with a skin modification stimulus, as well as applying a C-glycoside or derivative to a target area before treatment with a skin modification stimulus or after treatment with a skin modification stimulus.

As used herein, the expression “at least one” means one or more and thus includes individual components as well as mixtures/combinations.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, +/−1%, +/−0.5%, +/−0.1%, or +/−0.01%.

As used herein, the expressions “ranging from” and “between” are inclusive of the endpoints of the recited range(s).

As used herein, all ranges provided are meant to include every specific range within, and combination of sub ranges between, the given ranges. Thus, a range from 1-5, includes specifically 1, 2, 3, 4, and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “comprising” (and its grammatical variations) as used herein is used in the inclusive sense of “having” or “including” and not in the exclusive sense of “consisting only of.”

As used herein, “topical application” (and its grammatical variations) refers to the application of the compositions of the disclosure onto keratinous substrates such as skin.

As used herein, “reducing” or “improving” the appearance of skin means that the skin would show more signs of aging than it would have been in the absence of treatment according to methods disclosed herein.

As used herein, “reducing” inflammation means that although inflammation may occur, its appearance to the naked eye is less visible or noticeable than it would have been after treatment with a skin modification technique in the absence of treatment according to methods disclosed herein or the presence of inflammatory markers is less than there would have been after treatment with a skin modification technique in the absence of treatment according to methods disclosed herein.

Unless otherwise expressly stated, it is in no way intended that any method set forth herein be construed as requiring that its steps be performed in a specific order. Accordingly, where a method claim does not expressly recite an order to be followed by its steps or it is not otherwise specifically stated in the claims or descriptions that the steps are to be limited to a specific order, it is no way intended that any particular order be inferred.

The compositions and methods of the present disclosure can comprise, consist of, or consist essentially of the essential elements and limitations of the disclosure described herein, as well as any additional or optional ingredients, components, or limitations described herein or otherwise useful.

Several embodiments having been described above, the disclosure may be better understood by reference to examples. The following examples are intended for illustration purposes only, and should not be construed as limiting in any way.

### EXAMPLES

The following examples are intended to be non-limiting and explanatory nature only.

### Example 1: Cell Cultures

Surgical skin samples were taken from adults and subjected to the 0.25% trypsin dermis/epidermis separation method described in Rheinwald to obtain suspensions of normal human epidermal keratinocytes (NHEK) and dermal fibroblasts. See Rheinwald J. G., Green H.: Serial cultivation of strains of human epidermal keratinocytes: The formation of keratinizing colonies from single cells. Cell. 6 (3): 331-43. 1975. The separated normal human dermal fibroblasts were cultured in DMEM+10% fetal calf serum. The fibroblasts were cast onto a lower layer of proto-SoftSkin™ gel. The NHEK cell cultures were initiated using the 3T3 feeder-layer technique described in Rheinwald J. G., Green H.: Epidermal growth factor and the multiplication of cultured human epidermal keratinocytes. Nature 265:421-424 (1977).

### Example 2: Inflammation Model

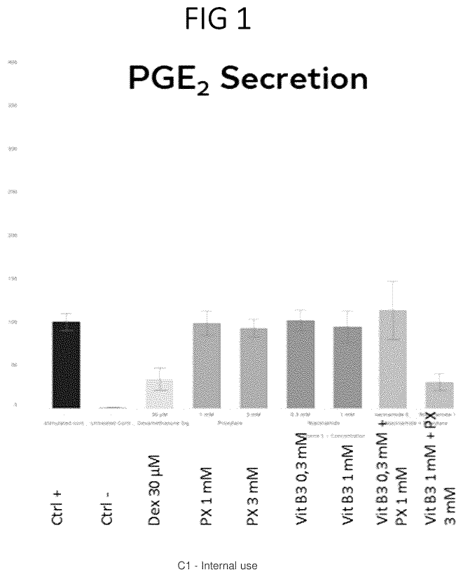

Fibroblasts as disclosed in Example 1 were stimulated with IL-1a and treated with proxylane (1 mM, 3 mM) and niacinamide (vitamin B3) (0.3 mM and 1 mM) alone and in combination to simulate inflammation in vitro. The amount of secreted PGE₂ was quantified. Results are shown in FIG. 1. It can be observed that 1 mM vitamin B3 and 3 mM proxylane synergistically reduce PGE₂ secretion in inflamed cell cultures, as compared to vitamin B3 or proxylane alone, and to as similar degree as the anti-inflammatory drug dexamethasone (30 μM).

### Example 3: αSMA Expression

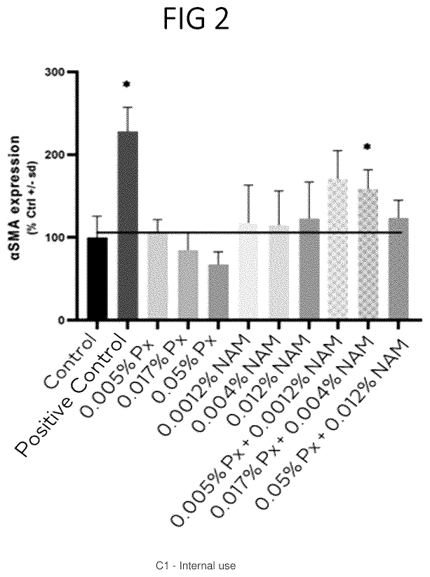

Fibroblasts as disclosed in Example 1 were treated with proxylane (0.005%, 0.017%, 0.05%) and niacinamide (0.0012%, 0.004%, and 0.012%) alone and in combination and stained against α-SMA via in situ immunolabeling. Expression was quantified through image analysis. Results are shown in FIG. 2. It can be observed that vitamin B3 and proxylane synergistically increase α-SMA expression in fibroblast cells, as compared to vitamin B3 or proxylane alone. The results demonstrate the ability of vitamin B3 and proxylane to stimulate α-SMA expression in fibroblasts, and thus the combination demonstrates wound contraction benefits and the ability to increase differentiation of fibroblasts into myofibroblasts in the tissue regeneration process.

### Example 4: Proliferation

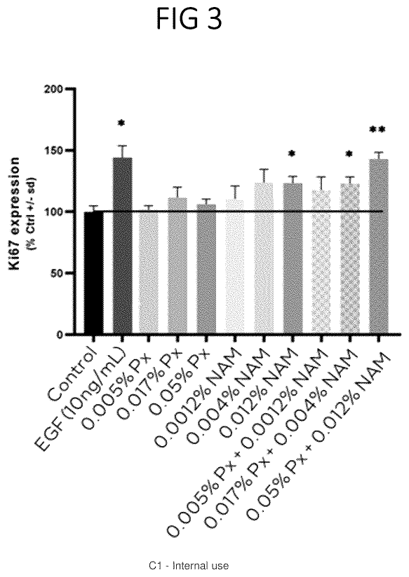

NHEKs as disclosed in Example 1 were treated with proxylane (0.005%, 0.017% and 0.05%) and niacinamide (0.0012%, 0.004%, and 0.012%) alone and in combination. Keratinocytes were stained against KI67 and expression was quantified. Results are shown in FIG. 3. It was demonstrated that vitamin B3 and proxylane were capable of stimulating cell proliferation of keratinocytes alone or in combination. It can be observed that 0.012% vitamin B3 and 0.05% proxylane synergistically increase α-SMA expression in keratinocytes.

### Example 5: sGAG Release

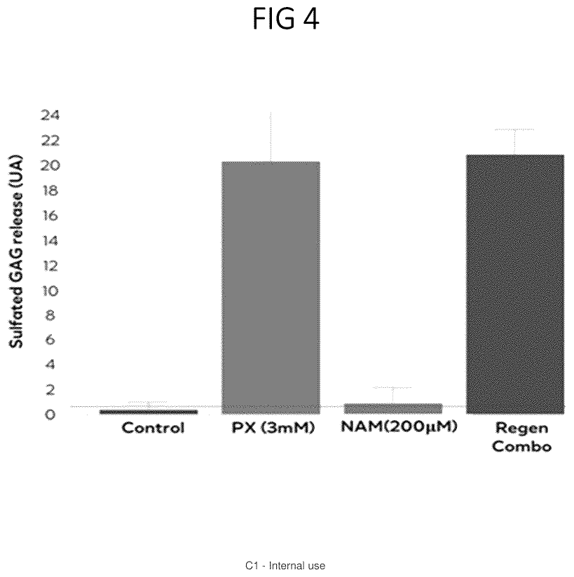

Full thickness reconstructed skin (T-Skin from Episkin) as disclosed in Example 1 was treated systemically with proxylane (3 mM) and niacinamide (200 μM) alone and in combination. SGAG release into the supernatant was quantified. Results are shown in FIG. 4. It can be observed that proxylane alone and in combination with vitamin B3 significantly increases sGAG secretion in fibroblasts.

### Example 6: PGE-2 Induced Inflammation and Melanin Synthesis

Normal Human Epidermal Melanocytes-Dark Pigment (Lot 1981687, Passage 3, ThermoFisher, Waltham, MA) were seeded in 6-well plates at a density of 100,000 cells per well. Cells were grown in culture medium supplemented with Gibo Human Melanocyte Growth Supplement-2 (ThermoFisher, Waltham, MA). Once reaching 70-80% confluence, the cell medium was removed and replaced with medium containing treatment conditions as demonstrated below:
Untreated Control
30 nM PGE₂
30 nM PGE₂+6 mM TXA (Positive Control)
30 nM PGE₂+10 mM (0.12%) Niacinamide.
30 nM PGE₂+10 mM (0.19%) Proxylane
30 nM PGE₂+10 mM (0.12%) Niacinamide+10 uM (0.19%) Proxylane

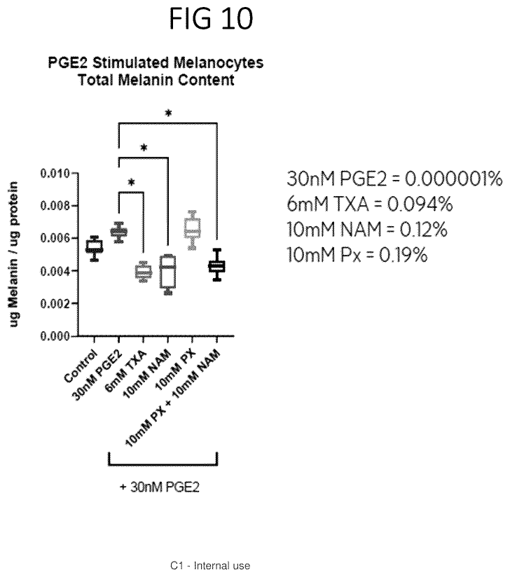

Cells were cultured for 10 days with treatment with renewal of treatment medium every other day with the exception of weekends. Following the 10 days, cells were trypsinized and the melanin was isolated using NaOH. A standard curve using commercially available melanin was used to calculate the average cellular melanin content using the recorded optical density of the isolated melanin using a microplate reader. Average cellular melanin content was normalized to total protein content. The results in FIG. 10 demonstrate that vitamin B3 alone and in combination with proxylane significantly decreases melanin production, demonstrating the efficacy of the ingredients in preventing inflammation-induced melanogenesis in melanocytes stimulated with PGE₂.

### Example 7: Neutrophil Elastase Inhibition Assay

A Neutrophil Elastase Inhibition Assay was performed according to manufacturer's instructions (MAK213, Sigma-Aldrich, MO). A 4× sample inhibitor solution (test compounds) was prepared, along with the inhibitor control, enzyme control (PBS), neutrophil elastase, and substrate solutions. The test compounds (proxylane, vitamin B3, and proxylane+vitamin B3), controls, and neutrophil elastase were added to a 96-well plate, protected from light, and incubated at 37° C. for 5 minutes. The substrate was then added and the fluorescence (FLU, λₑₓ=400 nm, λₑₘ=505 nm) was taken every minute for thirty minutes at 37° C. The slope of the plot (time vs FLU) was taken. Relative inhibition was calculated as follows:

$$\% Relative Inhibition = ( \frac{{Slope}_{Enzyme Control} - {Slope}_{sample}}{{Slope}_{Enzyme Control}} ) \times 100$$

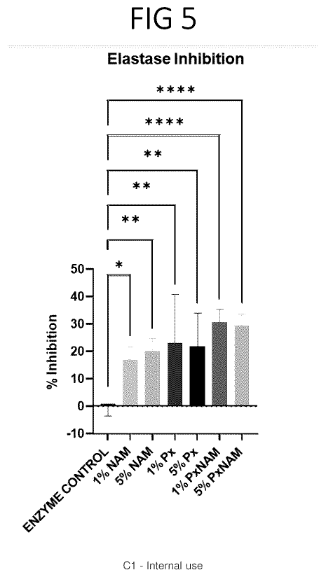

Results are shown in FIG. 5. The results demonstrate that vitamin B3 and proxylane alone and in combination inhibit elastase activity. It can be observed that vitamin B3 and proxylane synergistically inhibit elastase, as compared to vitamin B3 or proxylane alone.

### Example 8: Ex Vivo Microneedling Model

Fresh post-abdominoplasty normal human skin samples were acquired from BioIVT Inc. (Westbury, NY). Tissue was defatted and cleaned of blood residue. Tissue was then subjected to 5 passes using a microneedling pen (36-pin needles, Dr. Pen A6 Cartridges Tips, Dr. Pen Inc, San Jose, CA, USA) with a needle length of 1.5 mm. Following treatment, 1.2 cm diameter skin biopsies were created and cultured at air-liquid interface. Skin explants not subjected to microneedling served as the untreated control group. Control and microneedle control samples were cultured in Dulbecco's Modified Eagle's Medium (650 μL per well, DMEM with 10% Fetal Bovine Serum and 1% Penicillin-Streptomycin) at 37° C. and 5% CO₂. Treated microneedle samples (MN+Proxylane/Niacinamide) were cultured in DMEM with 10% Fetal Bovine Serum, 1% Penicillin-Streptomycin, 0.0009% Proxylane, and 0.2% Niacinamide. The media was changed every other day with the exception of weekends. Following a 6-day culture period, all biopsies were processed for histological and immunohistochemical analysis. Skin explants were processed for haematoxylin and eosin (H&E) staining and immunohistochemical staining against Filaggrin and TGM1 according to standard protocol (Histowiz, Brooklyn, NY).

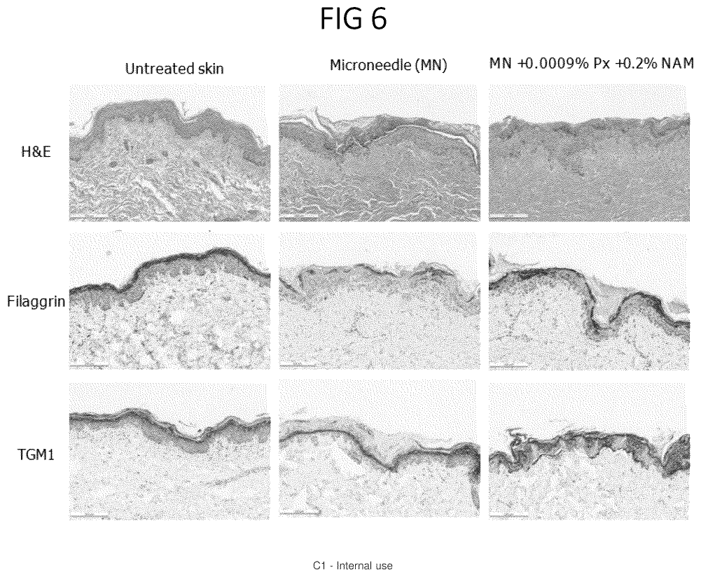

Results are shown in FIG. 6. It can be observed that vitamin B3 and proxylane increase the expression of Filaggrin and TGM1 after microneedling procedures. Thus, the results demonstrate that vitamin B3 and proxylane increase barrier regeneration and renewal and accelerate epidermal renewal and differentiation.

### Example 9: Non-Ablative Laser Study

An 8 week, double-blinded, randomized, controlled split-face study was conducted to evaluate the skin repair and anti-aging efficacy of the disclosed topical compositions vs. benchmark after full-face non-ablative laser treatment. Healthy subjects with (a) mild to severe global face skin dyschromia (facial dark spots/sunspots) (grade 3.5-8 on a modified Griffith scale of 0-9), with a difference≤1 between left and right sides of the face, (b) mild to severe visual skin roughness (grade 3.5-8 on internal scale 0-9), with a difference≤1 between left and right sides of the face, and (c) mild to severe crow's feet wrinkles (grade 2-5.6 on L'Oréal Aging Atlas scale 0-6, Caucasian or Asian) on both sides of the face, with difference≤1 between left and right sides of the face were included in the study.

A laser specialist under the supervision of a Board-certified Dermatologist performed full-face non-ablative fractional laser treatment on the test subjects, and post-laser evaluations. Composition A, disclosed in Table 1 below, and composition B, Aquafor Healing Ointment, were applied to different sides of the face in a randomized, double-blind matter immediately after the laser treatment. The compositions were applied twice daily for 56 days after laser treatment.

TABLE 1
Composition A

| | % Concentration |
|---|---|
| NIACINAMIDE | 3 |
| SODIUM HYALURONATE | 0.02 |
| PEG-100 STEARATE | 0.4 |
| SODIUM POLYACRYLATE | 0.8 |
| GLYCERYL STEARATE | 0.4 |
| BEESWAX/CERA ALBA | 2.73 |
| BEHENYL ALCOHOL | 0.6 |
| DIMETHICONE | 2.5 |
| ARACHIDYL ALCOHOL | 1.1 |
| ISONONYL ISONONANOATE | 1.0 |
| ADENOSINE | 0.04 |
| STEARIC ACID | 0.795 |
| PALMITIC ACID | 0.66 |
| MYRISTIC ACID | 0.045 |
| HYDROXYPROPYL TETRAHYDROPYRANTRIOL | 10.01 |
| ARACHIDYL GLUCOSIDE | 0.3 |
| ADDITIVES (thickeners, preservatives, vitamins, oils, | <10 |
| moisturizers, ceramides, pH adjusters, emulsifiers, | |
| sunscreen agents, chelators, etc.) | |
| SOLVENTS (water and non-aqueous solvents) | QS to 100 |

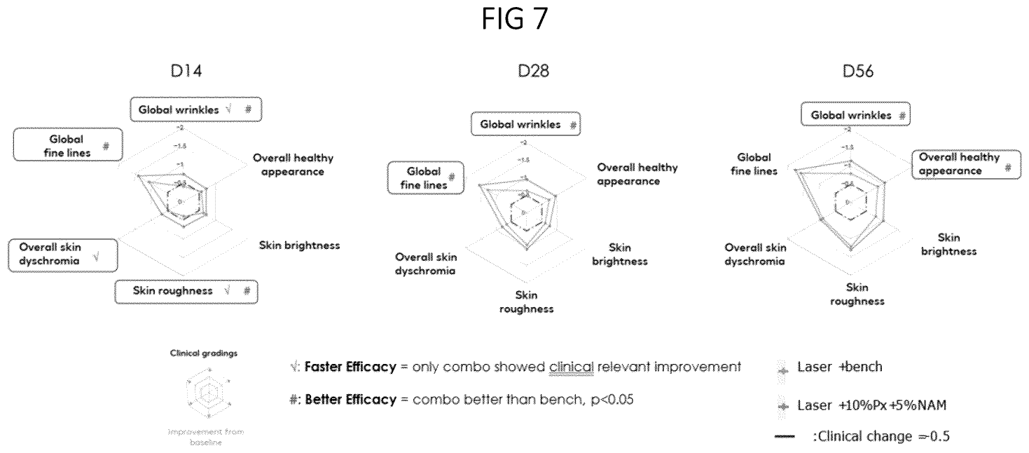

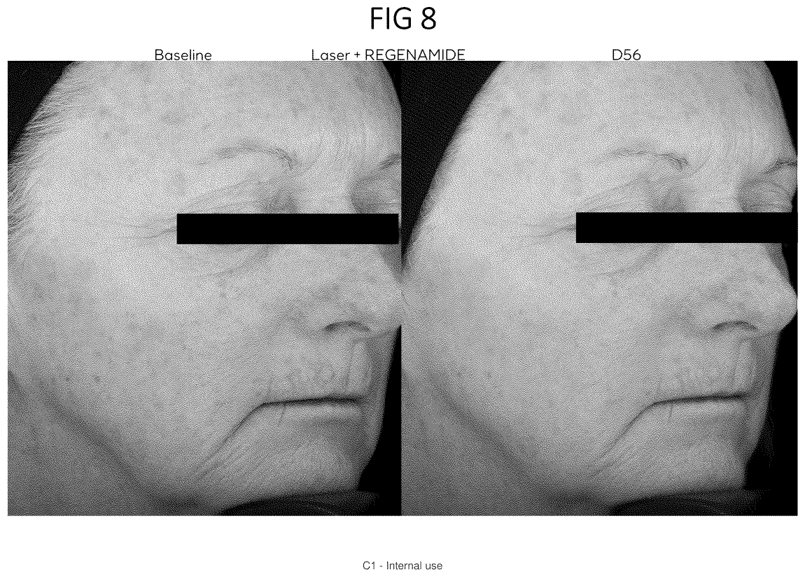

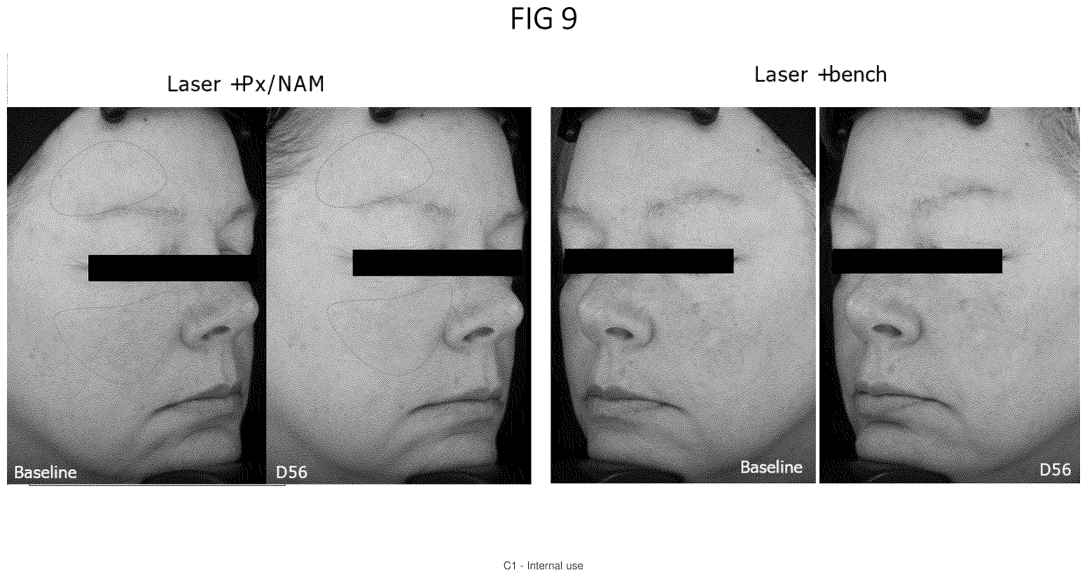

Following the 56-day trial period, experts evaluated and graded the efficacy of the topical compositions in promoting skin repair and improving skin dyschromia and aging signs, as well as maintaining the resurfacing effects when used after non-ablative fractional face laser resurfacing treatment on healthy subjects by clinical evaluation. FIG. 7 shows clinical evaluations of the skin healing process (D14, D28, and D56) separately on each side of the face (left, right) using the following objective anti-aging efficacy parameters: global wrinkles, overall healthy appearance, skin brightness, skin roughness, overall skin dyschromia, and global fine lines. The combination of vitamin B3 and proxylane significantly improved global wrinkles, fine lines, skin dyschromia, skin roughness, and overall skin health. The combination treatment also demonstrated efficacy in improving skin redness. In addition, the routine of the nonablative laser with proxylane and vitamin B3 imparted faster and better laser-treatment benefits compared to laser alone. FIGS. 8 and 9 show VISIA Digital photographs of each subject's face (DO and D56) (left 45°, right) 45° demonstrating improvements in global wrinkles, fine lines, skin dyschromia, skin roughness, and overall skin health in skin treated with vitamin B3 and proxylane.

1-44. (canceled)

45. A method for improving the appearance of skin, comprising:
(a) treating the skin with at least one skin modification stimulus,
(b)(1) applying at least one C-glycoside or derivative thereof to the skin, and
(b)(2) applying at least one vitamin B3 or derivative thereof to the skin.

46. The method of claim 45, wherein the step (b)(1) of applying at least one C-glycoside or derivative thereof to the skin and the step (b)(2) of applying at least one vitamin B3 or derivative thereof to the skin occur within about 60 days after the step (a) of treating the skin with at least one skin modification stimulus.

47. The method of claim 45, wherein the at least one C-glycoside comprises at least one C-xylopyranoside or derivative thereof.

48. The method of claim 45, wherein the at least one C-glycoside is chosen from C13-D-xylopyranoside-n-propan-2-one; Cα-D-xylopyranoside n-propan-2-one; C13-D-xylopyranoside-2-hydroxypropane; Cα-D-xylopyranoside-2-hydroxypropane; 1-(C13-D-fucopyranoside)-propane-2-one; 1-(Cα-D-fucopyranoside)-propan-2-one; 1-(C13-L-fucopyranoside)-propan-2-one; 1-(Cα-L-fucopyranoside)-propane-2-one; 2-one, 1-(C13-D-fucopyranoside)-2-hydroxypropane, 1-(Cα-D-fucopyranoside)-2-hydroxypropane; 1-(C13-L-fucopyranoside)-2-hydroxypropane; 1-(Cα-L-fucopyranoside)-2-hydroxypropane; 1-(C13-D-glucopyranosyl)-2-hydroxylpropane; 1-(Cα-D-glucopyranosyl)-2-hydroxylpropane; 1-(C13-D-galactopyranosyl)-2-hydroxylpropane; 1-(Cα-D-galactopyranosyl)-2-hydroxylpropane-1-(C13-D-fucofuranosyl) propan-2-one; 1-(Cα-D-fucofuranosyl)-propan-2-one; 1-(C13-L-fucofuranosyl)-propan-2-one; 1-(Cα-L-fucofuranosyl)-propan-2-one; C13-D-maltopyranoside-n-propan-2-one-Cα-D-maltopyranoside-n-propan-2-one-C13-D-maltopyranoside-2-hydroxypropane; Cα-D-maltopyranoside-2-hydroxypropane; derivatives thereof, or combinations thereof.

49. The method of claim 45, wherein the at least one C-glycoside is chosen from C13-D-xylopyranoside-2-hydroxypropane, Cα-D-xylopyranoside-2-hydroxypropane, or combinations thereof.

50. The method of claim 45, wherein the at least one vitamin B3 comprises niacin (nicotinic acid), niacinamide (nicotinamide), nicotinamide riboside, or combinations thereof.

51. The method of claim 45, wherein the at least one vitamin B3 comprises niacinamide.

52. The method of claim 45, wherein the at least one skin modification stimulus is chosen from one or more of a laser, plasma, radiofrequency, a high-intensity focused ultrasound, an electrical field, heat, a low-level light device, a needle, a microneedle, an abrasive element, or a chemical agent.

53. The method of claim 45, wherein the step of treating the skin with at least one skin modification stimulus comprises treating the skin with one or more of an ablative laser procedure, a non-ablative laser procedure, a microneedling procedure, a high-intensity focused ultrasound procedure, an electroporation procedure, a dermabrasion procedure, a microdermabrasion procedure, a plasma skin rejuvenation procedure, a chemical exfoliation procedure, or combinations thereof.

54. The method of claim 45, wherein the step (b)(1) of applying at least one C-glycoside or derivative thereof to the skin comprises applying a composition comprising at least one C-glycoside or derivative thereof and at least one carrier to the skin,
wherein the total amount of C-glycoside(s) and derivatives thereof present in the composition ranges from about 0.01% to about 50% by weight, relative to the total weight of the composition.

55. The method of claim 45, wherein the step (b)(2) of applying at least one vitamin B3 or derivative thereof to the skin comprises applying a composition comprising at least one vitamin B3 or derivative thereof and at least one carrier to the skin,
wherein the total amount of vitamin B3 (s) and derivatives thereof present in the composition ranges from about 0.0001% to about 50% by weight, relative to the total weight of the composition.

56. The method of claim 45, wherein the at least one C-glycoside or derivative thereof and the at least one vitamin B3 or derivative thereof are applied simultaneously.

57. The method of claim 45, wherein the weight ratio of the total amount the C-glycoside(s) and derivatives thereof to the total amount of the vitamin B3 and derivatives thereof applied to the skin in one simultaneous or consecutive application of each ranges from about 0.001:1 to about 10:1.

58. The method of claim 45, wherein (b)(1) applying at least one C-glycoside or derivative thereof to the skin and (b)(2) applying at least one vitamin B3 or derivative thereof to the skin are performed simultaneously by applying a composition comprising at least one C-glycoside or derivative thereof, at least one vitamin B3 or derivative thereof, and at least one carrier to the skin,
wherein the total amount of C-glycoside(s) and derivatives thereof present in the composition ranges from about 0.01% to about 50% by weight, relative to the total weight of the composition, and
wherein the total amount of vitamin B3 (s) and derivatives thereof present in the composition ranges from about 0.0001% to about 50% by weight, relative to the total weight of the composition.

59. The method of claim 45, comprising:
(a) treating skin with at least one skin modification stimulus to produce modified skin tissue, followed by applying:
(b)(1) at least one C-glycoside or derivative thereof to the modified skin tissue within about 24 hours of step (a), and
(b)(2) at least one vitamin B3 or derivative thereof to the modified skin tissue within about 24 hours of step (a),
wherein (b)(1) and (b)(2) are performed simultaneously or consecutively in any order.

60. The method of claim 59, wherein the application step (b)(1) is repeated at least one time at least about 18 hours after the first application.

61. The method of claim 60, wherein the application step (b)(2) is repeated at least one time at least about 18 hours after the first application.

62. The method of claim 60, wherein the application step (b)(2) occurs within about 12 hours of the application step (b)(1), in any order.

63. A kit comprising:
(i) a composition comprising at least one C-glycoside or derivative thereof and at least one carrier,
wherein the total amount of C-glycosides and derivatives thereof present in the composition ranges from about 0.01% to about 50% by weight, relative to the total weight of the composition, and
(ii) a composition comprising at least one vitamin B3 or derivative thereof and at least one carrier,
wherein the total amount of vitamin B3 and derivatives thereof present in the composition ranges from about 0.0001% to about 50% by weight, relative to the total weight of the composition.

64. A method for improving the outcome of aesthetic skin procedures, comprising:
(a) treating the skin with at least one skin modification stimulus thereby resulting in modified skin tissue,
(b)(1) applying at least one C-glycoside or derivative thereof to the modified skin tissue, and
(b)(2) applying at least one vitamin B3 or derivative thereof to the modified skin tissue.
